(19) United States
(12) Patent Application Publication
(10) Pub. No.: US 2022/0257728 A1
(43) Pub. Date: Aug. 18, 2022

(54) USE OF HISTONE DEUBIQUITINASE IN PREPARING DRUG FOR PREVENTING AGING AND TREATING AGING-RELATED DISEASES

(71) Applicant: JIANGUO WU, FOSHAN (CN)

(72) Inventors: JIANGUO WU, FOSHAN (CN); MINGFU TIAN, FOSHAN (CN); YUQING HUANG, FOSHAN (CN); KAILANG WU, FOSHAN (CN); QIUPING TAN, FOSHAN (CN)

(21) Appl. No.: 17/732,478

(22) Filed: Apr. 28, 2022

Related U.S. Application Data
(63) Continuation of application No. PCT/CN2020/110239, filed on Aug. 20, 2020.

(30) Foreign Application Priority Data
Aug. 12, 2020 (CN) 202010809325X

(51) Int. Cl. A61K 38/48; A61K 47/62; A61K 45/06; A61K 38/10

(52) U.S. Cl. CPC A61K 38/4813; A61K 38/10; A61K 45/06; A61K 47/62

(57) ABSTRACT

The present disclosure provides use of a histone deubiquitinase in preparing a drug for preventing aging and treating aging-related diseases and relates to the technical field of biological medicines. The histone H2A deubiquitinase MYSM1 has a nucleotide sequence shown as SEQ ID NO. 1. It is verified that MYSM1 protein deficiency causes significant aging of mice and induces the aging-related diseases; and the MYSM1 can promote DNA injury repair, prevent cellular senescence and suppress chronic inflammation. Proved by a result of using an adeno-associated virus expressing mouse-derived MYSM1 as a gene therapy, the MYSM1 may remarkably prolong the lifespan of the mice, reduce the process and the number of senescent cells, improve normal functions of tissues and organs, reduce occurrence and development of the aging-related diseases, which proves that the MYSM1 has a good application prospect in delaying aging and treating the aging-related diseases.

### TECHNICAL FIELD

The present disclosure belongs to the technical field of biomedicine and particularly relates to use of a histone deubiquitinase in preparing a drug for preventing aging and treating aging-related diseases.

### BACKGROUND

Delaying aging and prolonging lifespan are permanent dreams for people since ancient times. Aging is a common biological phenomenon in growth and development processes of bodies. Body aging is usually manifested by cell cycle disorders, excessive immune response and inflammatory response, resulting in immunological damages and dysfunction of tissues and organs and severe degenerative diseases. Therefore, it is important to study anti-aging molecular targets and use thereof in delaying aging and prolonging lifespan.

Many evidences support that cellular senescence is an important factor in occurrence and development of aging and age-related diseases [1-4]. The cellular senescence is a state in which immune injury or growth arrest of senescent cells is accompanied by release of immune factors and inflammatory factors under different stresses. The cellular senescence is characterized by secreting a large amount of proinflammatory factors, chemokines, extracellular matrix proteins, growth factors and proteases, which is known as senescence-associated secretory phenotype (SASP) [5-7]. The senescent cells exhibit marked functional changes. Such changes are driven by alterations in metabolism, chromatin organization and transcriptional regulation, which leads to production of the senescent cells and occurrence and development of related diseases [8-11]. Aging of body tissues and organs is mainly a process of continuously accumulated senescent cells with increase of age, and further leads to organ dysfunction and diseases. It is an essential anti-aging method and strategy that the senescent cells of the body are eliminated, so that the vitality of tissues and organs is restored, and aging is delayed. Without a doubt, cellular senescence is a key marker of body aging and is important in development of the age-related diseases [12]. Unfortunately, mechanisms of aging have not been fully disclosed. In the previous work, a cytokine MYSM1 for suppressing immune response and cellular senescence is found for the first time and the action mechanism of the cytokine MYSM1 is elucidated. Thus, the MYSM1 may be used as a drug for preventing aging and treating the aging-related diseases.

Gene therapy is an essential and effective novel therapeutic means and means that an exogenous normal gene is introduced into a target cell to treat or relieve diseases caused by defective and abnormal genes and then achieve the purpose of treating the diseases. Gene therapy further comprises a technical application in the aspect of transgene, that is, an exogenous gene is inserted into a proper receptor cell of a patient by a transgenic technology, such that a product of the foreign gene can treat diseases. The gene therapy has been widely used in treating various diseases. By August, 2019, organizations such as the European Medicines Agency (EMA), the U.S. Food and Drug Administration (FDA) and the National Medical Products Administration (NMPA) have approved at least 13 gene therapy products for marketing. Over 2,500 cell and gene therapies are undergoing clinical trials [13]. The gene therapy will develop towards safer and more efficient vector engineering. Personalized gene therapy can be performed on a patient by being combined with various existing strategies of viral vectors and gene engineering. In summary, development of the gene therapy has created exciting new therapeutic opportunities for diseases that have heretofore been incurable. Therefore, it is of important clinical value to develop a new gene therapy target for preventing and treating aging and treating related degenerative diseases.

### REFERENCES

[1] Herbig U, Ferreira M, Condel L, Carey D, Sedivy J M. Cellular senescence in aging primates. Science 2006; 311: 1257.
[2] Campisi J. Aging, cellular senescence, and cancer. Annu Rev Physiol 2013; 75: 685-705.
[3] van Deursen, J M. The role of senescent cells in ageing. Nature 2014; 509: 439-446.
[4] Childs B G, Dunk M, Baker D J, van Deursen J M. Cellular senescence in aging and age-related disease: from mechanisms to therapy. Nat Med 2015; 21: 1424-1435.
[5] Kuilman T, Michaloglou C, Mooi W J, Peeper D S. The essence of senescence. Genes Dev 2010; 24(22): 2463-2479.
[6] Wajapeyee N, Serra R W, Zhu X, Mahalingam M, Green M R. Oncogenic BRAF induces senescence and apoptosis through pathways mediated by the secreted protein IGFBP7. Cell 2008; 132: 363-374.
[7] Acosta J C, et al. A complex secretory program orchestrated by the inflammasome controls paracrine senescence. Nat Cell Biol 2013; 15: 978-990.
[8] Baker D J, et al. Clearance of p16Ink4a-positive senescent cells delays ageing-associated disorders. Nature 2011; 479: 232-236.
[9] Krtolica A, Parrinello S, Lockett S, Desprez P Y, Campisi J. Senescent fibroblasts promote epithelial cell growth and tumorigenesis: A link between cancer and aging. Proc Natl Acad Sci USA 2001; 98: 12072-12077.
[10] Krizhanovsky V, et al. Senescence of activated stellate cells limits liver fibrosis. Cell 2008; 134: 657-667.
[11] Coppe J P, Desprez P Y, Krtolica A, Campisi J. The senescence-associated secretory phenotype: The dark side of tumor suppression. Annu Rev Pathol 2010; 5: 99-118.
[12] Sharpless N E, Sherr C J. Forging a signature of in vivo senescence. Nat Rev Cancer 2015; 15: 397-408.
[13] High K A and Roncarolo M G. Gene Therapy. N Engl J Med 2019; 381: 455-464.

### SUMMARY

For this purpose, an objective of the present disclosure is to provide use of a histone deubiquitinase in preparing a drug for preventing aging and treating aging-related diseases. MYSM1 may remarkably prolong the lifespan of mice, reduce the process and the number of senescent cells, improve normal functions of tissues and organs, and reduce occurrence and development of the aging-related diseases.

In order to realize the aforementioned objective of the present disclosure, the present disclosure provides the following technical solutions.

The present disclosure provides use of a gene expressing a histone H2A deubiquitinase MYSM1 in preparing a drug for preventing aging and treating aging-related diseases, where the gene has a nucleotide sequence shown as SEQ ID NO. 1.

Preferably, the MYSM1 may have an amino acid sequence shown as SEQ ID NO. 2.

The present disclosure provides an adeno-associated virus expressing a histone H2A deubiquitinase MYSM1, where the histone H2A deubiquitinase MYSM1 has an amino acid sequence shown as SEQ ID NO. 2.

The present disclosure provides a gene therapy kit for preventing aging and treating aging-related diseases, where the kit includes the adeno-associated virus.

The present disclosure provides a drug for preventing aging and treating aging-related diseases, where an active ingredient of the drug includes a histone H2A deubiquitinase MYSM1 and the histone H2A deubiquitinase MYSM1 has an amino acid sequence shown as SEQ ID NO. 2.

The present disclosure provides use of a gene expressing a histone H2A deubiquitinase MYSM1 in preparing a drug for preventing aging and treating aging-related diseases, where the gene has a nucleotide sequence shown as SEQ ID NO. 1. An adeno-associated virus (AAV) expressing mouse-derived and/or human-derived MYSM1 is used as a gene therapy means for preventing aging. Meanwhile, the MYSM1 protein and a polypeptide thereof (including the MYSM1 protein and the polypeptide thereof obtained by various technologies and approaches such as expression, purification, synthesis or modification) are used for performing the use of preventing aging. A result shows that MYSM1 protein deficiency causes significant aging of mice and induces the aging-related diseases. A mechanism study shows that the MYSM1 promotes DNA injury repair, prevents cellular senescence and suppresses chronic inflammation. Proved by a result of using an adeno-associated virus expressing the mouse-derived MYSM1 as a gene therapy, the MYSM1 may remarkably prolong the lifespan of mice, reduce the process and the number of senescent cells, improve normal functions of tissues and organs, and reduce occurrence and development of the aging-related diseases. In the present disclosure, the MYSM1 protein is effectively used for preventing aging and treating the aging-related diseases and has very significant effects; and thus the MYSM1 has a very good application prospect in delaying aging and treating the aging-related diseases.

### BRIEF DESCRIPTION OF THE DRAWINGS

FIG. 1 is a diagram that MYSM1 suppresses cellular senescence;

FIG. 2 is a molecular mechanism diagram of MYSM1 in suppressing cellular senescence;

FIG. 3 is a diagram that MYSM1 knock-out induces accelerated aging of body; and

FIG. 4 is a diagram that MYSM1 gene therapy significantly delays aging of a body and suppresses occurrence and development of aging-related diseases.

### DETAILED DESCRIPTION

The present disclosure provides use of a gene expressing a histone H2A deubiquitinase MYSM1 in preparing a drug for preventing aging and treating aging-related diseases, characterized in that the gene has a nucleotide sequence shown as SEQ ID NO. 1. The MYSM1 of the present disclosure may preferably have an amino acid sequence shown as SEQ ID NO. 2. In the present disclosure, the histone H2A deubiquitinase (2A-DUB) is also called Myb-like, SWIRM and MPN domain-containing protein 1 (MYSM1). The MYSM1 may promote DNA injury repair, prevent cellular senescence, suppress chronic inflammation, prevent occurrence and development of tumors, etc.

The present disclosure further provides an adeno-associated virus expressing a histone H2A deubiquitinase MYSM1, where the histone H2A deubiquitinase MYSM1 has an amino acid sequence shown as SEQ ID NO. 2. The present disclosure has no particular limitation on a viral vector of the adeno-associated virus (AAV) and a viral vector and/or other gene therapy vectors conventional in the art may be used. In the embodiments of the present disclosure, in order to conveniently illustrate the specific effects of the MYSM1, an adeno-associated virus (AAV) expressing mouse-derived MYSM1 is used as gene therapy to treat aged mice. A result of gene therapy proves that the MYSM1 may remarkably reduce the process and the number of senescent cells, improves normal functions of tissues and organs, reduces occurrence and development of aging-related diseases and prolongs the lifespan of mice.

The present disclosure further provides a gene therapy kit for preventing aging and treating aging-related diseases, where the kit includes the adeno-associated virus. The adeno-associated virus of the present disclosure may express the MYSM1 in vitro and the MYSM1 protein obtained after separation and purification has high activity. When the kit is used to perform gene therapy for preventing aging and treating the aging-related diseases, the MYSM1 is used as a target of gene therapy and has an obvious anti-aging effect.

The present disclosure further provides a drug for preventing aging and treating aging-related diseases, where an active ingredient of the drug includes a histone H2A deubiquitinase MYSM1, and the histone H2A deubiquitinase MYSM1 has an amino acid sequence shown as SEQ ID NO. 2. In the present disclosure, the MYSM1 protein and a polypeptide thereof (including the MYSM1 protein and the polypeptide thereof obtained by various technologies and approaches such as expression, purification, synthesis or modification) are used for performing the use of preventing aging and treating the aging-related diseases.

The use of the histone deubiquitinase provided by the present disclosure in preparing the drug for preventing aging and treating the aging-related diseases will be described in detail below with reference to the embodiments, but it should not be understood as limiting the protection scope of the present disclosure.

### Embodiment 1

Correlations of MYSM1 with age and aging were determined. Analysis on mouse samples: mice of different ages were taken, and an expression of the MYSM1 was detected. Primary cell experiment: with mouse embryonic fibroblasts (MEFs) isolated from C57BL/6 mice as a model, and a senescence inducer etoposide (ETO) was used to stimulate the MEFs, and an expression of the MYSM1 was detected. Molecular and cell biology research: the senescence inducer etoposide (ETO), hydrogen peroxide (H₂O₂), etc. were used to stimulate human embryonic lung fibroblasts (WI-38) and primary C57BL/6 mouse embryonic fibroblasts (MEFs), and expressions and secretion of immune factors, inflammatory factors and aging-associated molecules were detected and analyzed.

The specific steps were as follows:

(A-C) C57BL/6 MEFs were stimulated with the ETO or doxorubicin hydrochloride (DOX) (A). Kidneys (B) and lungs (C) were harvested from 2-month-old and 22-month-old wild-type (WT) mice (n=3 per group). mRNAs of a Mysm1 gene were detected by RT-PCR. MYSM1 and GAPDH proteins were detected by immunoblotting. (D-F) WT or Mysm1^{-/-} MEFs were pretreated for 48 hours to induce senescence [Mysm1 knock-out C57BL/6 mice (Mysm1^{-/-}) were established by using C57BL/6 mice with a clean background, a stable strain and easiness in reproduction as a model]. Cell proliferation ability (D) was determined by a CCK8 kit. Colony formation (E) was analyzed by crystal violet staining. The activity (F) of a cellular senescence marker senescence associated-β-galactosidase (SA-β-gal) was determined by X-Gal staining. (G) The 2-month-old WT and Mysm1^{-/-} mice were exposed to ionizing radiation (n=3 per group). The activity of the SA-β-Gal in lungs and livers after stimulation was detected by immunohistochemistry. (H-K) The WT or Mysm1^{-/-} MEFs were continuously cultured for different generations (P) or different days (D). Population doubling levels (H) were determined. Cell proliferation ability (I) was determined. Colony formation (J) was analyzed. The activity (K) of the SA-β-Gal was determined by X-Gal staining. (L-N) The activity of the SA-β-Gal in the lungs (L), livers (M) and spleens (N) of 2- and 6-month-old WT and Mysm1^{-/-} mice was measured by IHC analysis (n=3 per group).

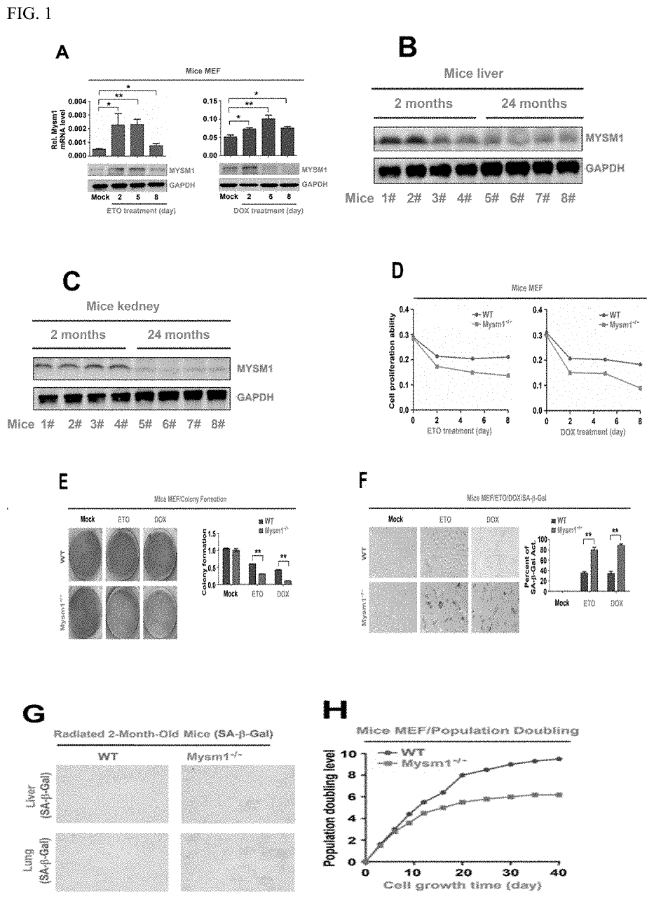

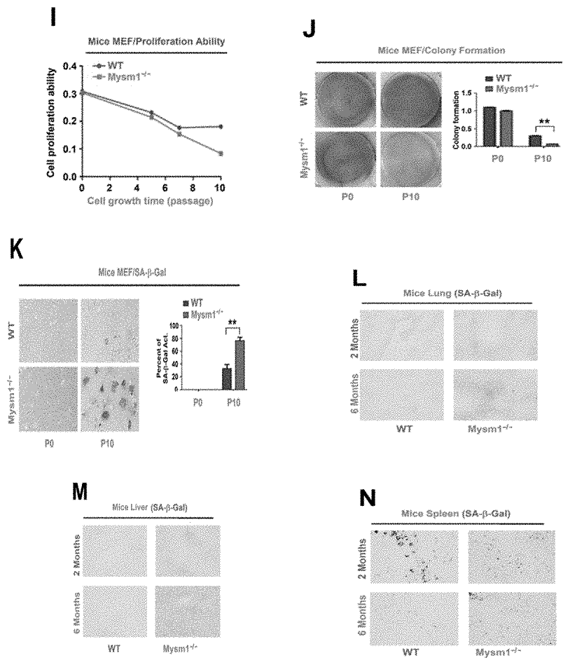

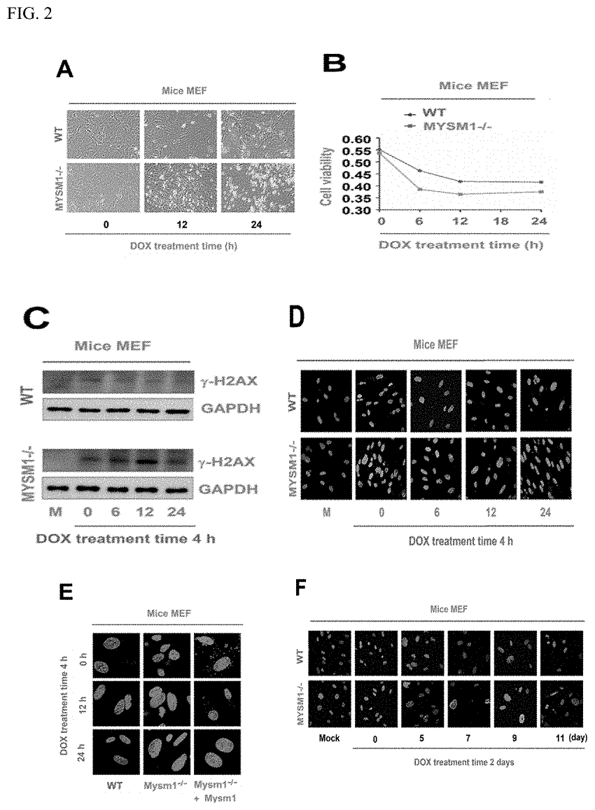

The results were shown in FIG. 1. The MYSM1 was significantly decreased in aged tissues. Meanwhile, knock-out of the MYSM1 significantly promoted drug-induced cellular senescence as well as cell senescence in the natural state, indicating that the MYSM1 suppressed the cellular senescence.

### Embodiment 2

### Molecular Mechanism of MYSM1 in Suppressing Cellular Senescence

Correlations between MYSM1 and a DNA damage were determined. Analysis on mouse samples: mice of different ages were taken, and expressions of DNA damage signals in Mysm1 wild-type mice (C57BL/6 WT) and Mysm1 knock-out mice (C57BL/6 Mysm1^{-/-}) were detected. Primary cell experiment: with MEFs isolated from C57BL/6 mice as a model, a senescence inducer etoposide (ETO) was used for stimulating the MEFs, and cell viability was detected. The DNA damage was detected. Molecular and cell biology research: the senescence inducer ETO, hydrogen peroxide (H₂O₂), etc. were used to stimulate human embryonic lung fibroblasts (WI-38) and primary C57BL/6 mouse embryonic fibroblasts (MEFs), a response of a cell damage repair system was detected, and a mechanism of the MYSM1 regulating the damage was illustrated.

The steps were as follows: (A and B) wild-type (WT) or Mysm1^{-/-} MEFs were treated with doxorubicin hydrochloride (DOX). Cytotoxicity (A) was analyzed based on cytopathy. Cell viability was determined by a CCK8 kit. (C-E) WT and Mysm1^{-/-}MEFs induced the DNA damage by using a stimulant. γ-H2AX and GAPDH proteins were detected by WB (C). The γ-H2AX protein was detected with an anti-γ-H2AX antibody and an activation level of the γ-H2AX was observed and confirmed under a confocal microscope (D and E). (F) The MEFs were treated with the DOX for 2 days to induce the DNA damage. The γ-H2AX protein was detected with the anti-γ-H2AX antibody and an activation level of the γ-H2AX was observed under the confocal microscope. (G) 2-month-old WT and Mysm1^{-/-} mice were exposed to radiation (n=3 per group). The γ-H2AX in a lung tissue was determined by an IHC method. (H) MEFs were passaged in serial culture, and the DNA damage was induced. The γ-H2AX was detected and visualized under the confocal microscope. (I) The MEFs were continuously cultured for 10 passages, and the DNA damage was induced. The γ-H2AX protein was detected with the anti-γ-H2AX antibody, and an activation level of the γ-H2AX was observed under the confocal microscope. (J-K) Livers, spleens, and lungs were taken from 10-month-old WT and Mysm1^{-/-} mice (n=3 per group), and the γ-H2AX protein was detected with the anti-γ-H2AX antibody. (M-P) Human HEK293T cells (M, N) or mouse MEFs (0, P) were co-transfected with a human HR reporter system (M,O) or an NHEJ reporter system (N, P). The efficiency of the HR (M) and the NHEJ (N) was determined.

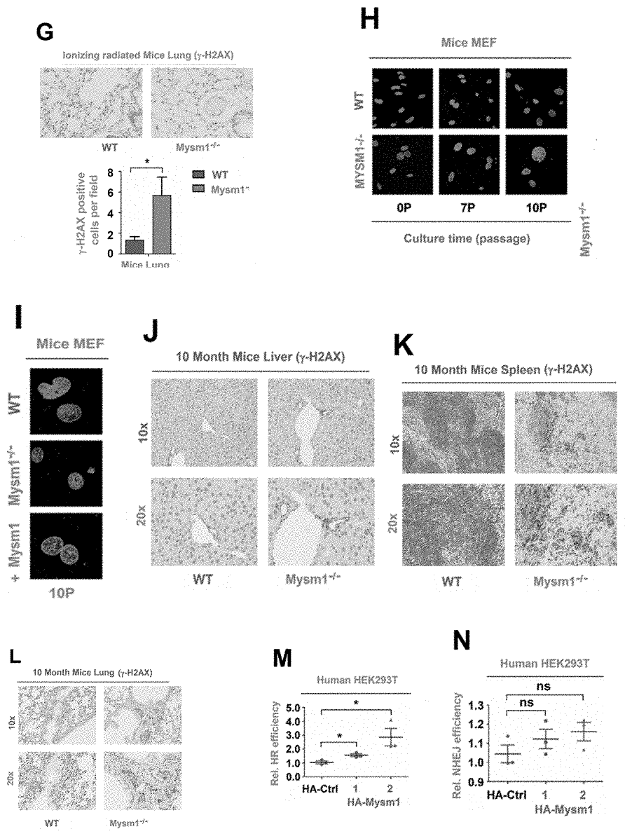

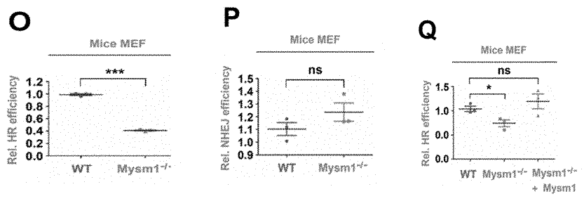

The results were as shown in FIG. 2. The MYSM1 promoted DNA injury repair, prevented cellular senescence and suppressed chronic inflammation.

### Embodiment 3

Causing Accelerated Aging of Body with MYSM1 Knock-Out

A C57BL/6 mouse model was used to establish MYSM1 knock-out mice. The gene knock-out mice were stimulated with a senescence inducer etoposide and ionizing radiation. Meanwhile, natural aging mice were studied, and expressions of immune factors and inflammatory factors in tissues and organs and expressions of aging-related molecules were detected and analyzed to confirm a function of MYSM1 in regulating the lifespan and tissue damages of mice. Aged mice were taken for analysis on activation of immune pathways in tissues and organs in a natural aging model, and then the aging condition and an organ damage mechanism were confirmed. It was confirmed that the MYSM1 knock-out had promoted aging.

The steps were as follows: (A-D) Image analysis on body size (A), body shape (B), chest (C) and head (D) in 10-month-old WT and Mysm1^{-/-} mice. (E) Analysis on body lengths (cm) of 2-month-old WT and Mysm1^{-/-} mice (n=7 per group). (F) Analysis on body weights of WT and Mysm1^{-/-} mice (n=3 per time point). (G) Images of eyes of 6-month-old WT and Mysm1^{-/-} mice. (H) Incidence of cataract and other eye diseases in WT mice (n=16) and Mysm1^{-/-} mice (n=17). (I) Images of spleens of 6-month-old WT and Mysm1^{-/-} mice (n=5 per group). (J) Images of livers of 2-month-old WT and Mysm1^{-/-} mice (n=5 per group). (K) Images of livers of 6-month-old WT and Mysm1^{-/-} mice (n=5 per group). (L) Survival rate of WT mice (n=6) and Mysm1^{-/-} mice (n=7). (M-Q) Masson's trichrome staining images of kidneys (M), hearts (N), muscles (O), lungs (P) and livers (Q) of 6-month-old WT and Mysm1^{-/-} mice (n=3 per group). (R) PAS histochemical staining analysis on kidneys of 6-month-old WT and Mysm1^{-/-} mice (n=3 per group).

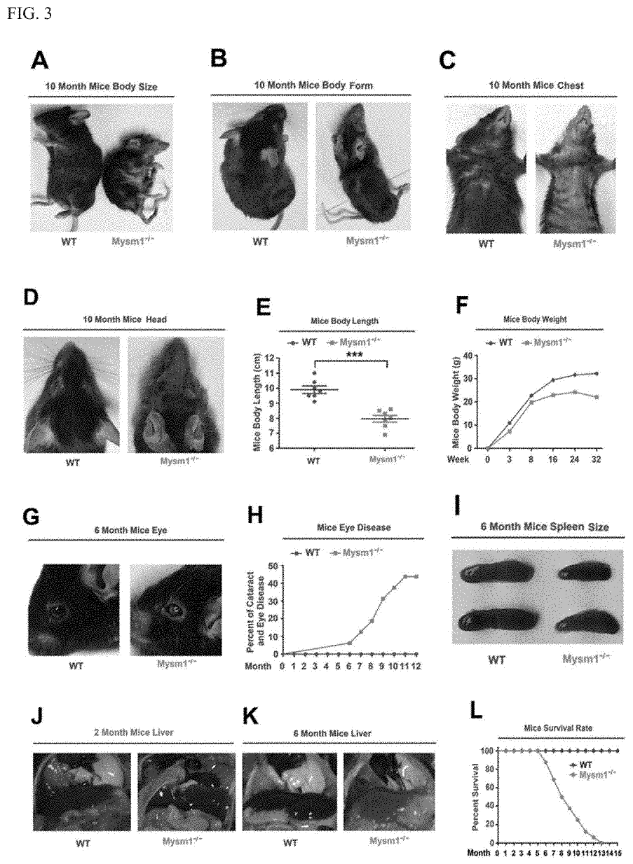

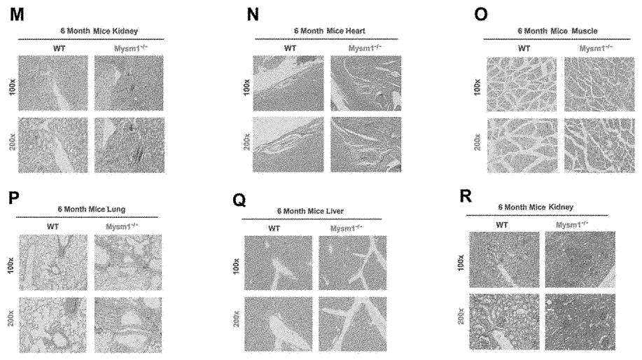

The results were as shown in FIG. 3 that the C57BL/6 mouse model was used to establish the MYSM1 knock-out mice. The expressions of immune factors and the inflammatory factors in tissues and organs and the expressions of the aging-related molecules were detected and analyzed to confirm the function of the MYSM1 in regulating lifespan and tissue damage of mice. Besides, the MYSM1 knock-out promoted aging of a body.

### Embodiment 4

Significant Delaying in Aging of Body and Suppression in Occurrence and Development of Aging-Related Diseases with MYSM1 Gene Therapy

Small animal experiment: A C57BL/6 mouse model was used, 16-month-old mice were injected with an AAV-9-related MYSM1-expressing virus and a control virus, and expressions of immune factors and inflammatory factors in tissues and organs and expressions of aging-related molecules were detected and analyzed to confirm functions of MYSM1 therapy in prolonging the lifespan and repairing a tissue damage of mice. It was confirmed that the MYSM1 might be used as a target for gene therapy to suppress aging of a body.

The steps were as follows: (A-D) treatment on 14-month-old WT mice with AAV9-Ctrl (n=4) or AAV9-Mysm1 (n=5) for 2 months. Images of 16-month-old mice after treatment (A, top). Determination on the content of p16 and p21 proteins in kidneys and hearts by a WB method (A, middle and bottom). Determination on the levels of IL-6 (left), p16 (middle) and p21 (right) proteins in livers (B), kidneys (C) and hearts (D) of the mice by an IHC method. (E-Q) Treatment on 16-month-old WT mice with AAV9-Ctrl or AAV9-Mysm1 for 6 months (n=5 per group). Images of mice before and after treatment (E, top), images of mouse hair after treatment (E, middle), and analysis on mouse hair changes after treatment (E, bottom). A survival rate of the treated mice (F). Eye images and frequency analysis of cataracts and other eye diseases of treated mice (right) (G). (H-J) Detection on γ-H2AX in livers, kidneys and hearts of the treated mice by IHC. (K-M) Images of livers, kidneys and hearts, subjected to H&E staining, of the mice. (N-P) Analysis on livers, kidneys and hearts of the treated mice by Masson's trichrome staining. (Q) PAS analysis on mouse kidneys.

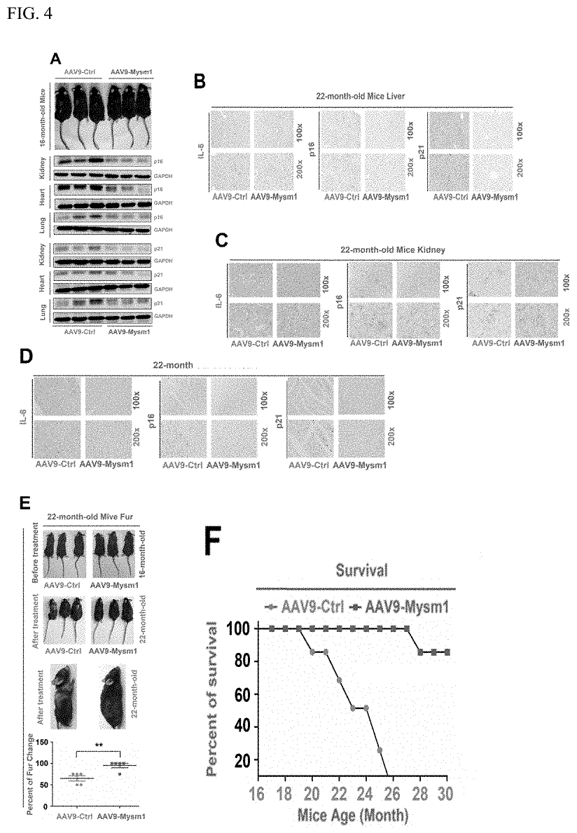

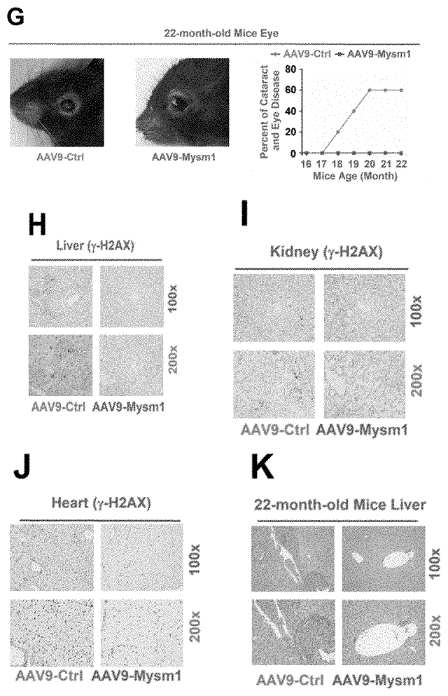

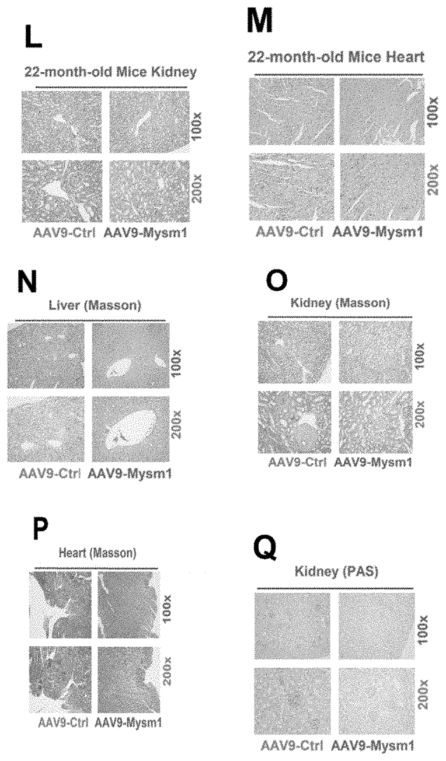

From the results, as shown in FIG. 4, it was confirmed that the MYSM1 treatment prolonged the lifespan and repaired tissue damages of the mice. It was confirmed that the MYSM1 might be used as a target for gene therapy to suppress aging of a body.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. Use of a gene expressing a histone H2A deubiquitinase MYSM1 in preparing a drug for preventing aging and treating aging-related diseases, wherein the gene has a nucleotide sequence shown as SEQ ID NO. 1.

2. The use according to claim 1, wherein the MYSM1 has an amino acid sequence shown as SEQ ID NO. 2.

3. A gene therapy kit for preventing aging and treating aging-related diseases, wherein the kit comprises the adeno-associated virus expressing a histone H2A deubiquitinase MYSM1, the histone H2A deubiquitinase MYSM1 has an amino acid sequence shown as SEQ ID NO. 2.

4. A drug for preventing aging and treating aging-related diseases, wherein an active ingredient of the drug comprises a histone H2A deubiquitinase MYSM1, and the histone H2A deubiquitinase MYSM1 has an amino acid sequence shown as SEQ ID NO. 2.


SEQUENCE LISTING (2 sequences)

SEQ ID NO: 1 - DNA, 2487 bp, Artificial Sequence
  gene expressing a histone H2A deubiquitinase MYSM1
atggcggctgaagaggcggatgtggatatcgaaggggacgtggtagcggcggcgggggcacagccaggaagtggtgaaaatacagcatcagttttacaaaaagatcactatcttgattcatcttggagaacagagaatggccttattccttggaccttggataacaccatcagtgaagagaacagagctgttattgagaaaatgttgttggaagaagaatattatttatctaaaaaatcacaaccggaaaaagtctggcttgatcaaaaggaagatgataaaaaatacatgaagagtctgcagaaaacagcaaaaatcatggtacactctcctacaaaaccagccagttactcagtaaagtggacgatagaagaaaaagagctgtttgaacaagggctggctaaatttggccgaagatggaccaaaatttcaaagctaattggaagccgcactgttttacaagtgaagagttatgcaagacagtattttaaaaataaggtcaaatgcggtctggataaagaaacaccaaatcagaagaccggccataatcttcaagttaaaaatgaagataaagggacaaaggcatggacaccatcatgtttaaggggacgtgctgatcccaacttgaatgctgtaaaaattgaaaagttatctgatgatgaagaagtagacatcacagatgaggtggacgagttgtcttctcaaacaccccagaagaattctagcagtgatctcttgttagactttcctaatagtaaaatgcatgaaaccaatcaaggagaattcattacttctgacagccaggaagctctcttttctaagtcttccaggggctgtcttcaaaatgaaaagcaagatgaaacactttcaagctcagaaattacactgtggactgagaaacagagcaatggtgacaaaaaatcaattgaattaaatgaccagaaatttaatgaattgattaaaaactgcaacaagcatgatggaaggggaataatagttgatgccaggcagttgccttctccagagccttgtgaaattcagaaaaatttgaatgataatgaaatgctttttcattcttgccaaatggtagaggaaagccatgaggaagaagagcttaagccaccagaacaggaaatagaaatagatagaaatatcattcaagaagaagaaaaacaagcaattcctgagttttttgaggggcgccaagctaaaacaccagaacgctatttgaaaattagaaattatattttggatcaatgggagatatgcaaaccaaaatacttaaataagacctcagtacgtcctggcctgaagaactgtggagatgttaattgtattggacggattcatacatacctcgaattgataggagcaatcaattttggatgtgaacaggctgtgtataataggccacaaacagttgacaaagtacgaatcagagacagaaaagatgcagtagaagcataccaacttgcccagcgtctgcagtctatgcgtacaaggagacgtagggtccgagacccatggggaaactggtgtgatgcaaaggacttagaaggacaaacgtttgagcatctctctgctgaggagttggcaaaaagaagagaagaggaaaaaggcagacctgttaaatctttaaaagtgccaagaccaacaaaaagctcgtttgatcccttccaactgataccttgtaatttttttagtgaagaaaagcaggagccatttcaggtgaaagtggcttcagaagcacttttaataatggatttgcatgctcatgtttctatggcagaagtgattggtctgttaggaggaagatactcagaagttgataaagtagttgaagtctgtgcagcagaaccatgtaacagtctgagtacaggactacagtgtgagatggatcctgtatcacaaacacaggcctcagaaaccttggctgttagaggcttcagtgttattggatggtatcattctcatcctgcttttgatcctaatccttccttacgagatattgacacacaagctaaataccagagttacttctccagaggaggtgcaaagttcattgggatgattgttagtccctataatcgaaataatcccttaccatattctcagattacctgcctggttataagtgaggaaattagcccagatggctcttatcgcttaccttacaaatttgaagtacagcagatgttagaagaacctcagtggggattagtatttgaaaagacaagatggataatagaaaaatacaggctctcccatagcagcgtccccatggataaaatctttcgccgggattctgacctgacttgtttgcagaaacttttggagtgtatgaggaagactctgagcaaagtgaccaattgctttatggctgaagaattcttgactgaaatagaaaatttgttcctttccaattataaaagcaaccaagagaatggagtaaccgaagagaactgtacaaaggaattgttaatgtga

SEQ ID NO: 2 - protein, 828 aa, Artificial Sequence
  MYSM1
MAAEEADVDIEGDVVAAAGAQPGSGENTASVLQKDHYLDSSWRTENGLIPWTLDNTISEENRAVIEKMLLEEEYYLSKKSQPEKVWLDQKEDDKKYMKSLQKTAKIMVHSPTKPASYSVKWTIEEKELFEQGLAKFGRRWTKISKLIGSRTVLQVKSYARQYFKNKVKCGLDKETPNQKTGHNLQVKNEDKGTKAWTPSCLRGRADPNLNAVKIEKLSDDEEVDITDEVDELSSQTPQKNSSSDLLLDFPNSKMHETNQGEFITSDSQEALFSKSSRGCLQNEKQDETLSSSEITLWTEKQSNGDKKSIELNDQKFNELIKNCNKHDGRGIIVDARQLPSPEPCEIQKNLNDNEMLFHSCQMVEESHEEEELKPPEQEIEIDRNIIQEEEKQAIPEFFEGRQAKTPERYLKIRNYILDQWEICKPKYLNKTSVRPGLKNCGDVNCIGRIHTYLELIGAINFGCEQAVYNRPQTVDKVRIRDRKDAVEAYQLAQRLQSMRTRRRRVRDPWGNWCDAKDLEGQTFEHLSAEELAKRREEEKGRPVKSLKVPRPTKSSFDPFQLIPCNFFSEEKQEPFQVKVASEALLIMDLHAHVSMAEVIGLLGGRYSEVDKVVEVCAAEPCNSLSTGLQCEMDPVSQTQASETLAVRGFSVIGWYHSHPAFDPNPSLRDIDTQAKYQSYFSRGGAKFIGMIVSPYNRNNPLPYSQITCLVISEEISPDGSYRLPYKFEVQQMLEEPQWGLVFEKTRWIIEKYRLSHSSVPMDKIFRRDSDLTCLQKLLECMRKTLSKVTNCFMAEEFLTEIENLFLSNYKSNQENGVTEENCTKELLM